(19) United States
(12) Patent Application Publication
(10) Pub. No.: US 2020/0111555 A2
(43) Pub. Date: Apr. 9, 2020

(54) SYNCHRONIZATION OF HIERARCHICAL DATA

(71) Applicants: Hermann HERTER, Leverkusen (DE); Jaspreet Singh MAROK, Köln (DE); Andrej CEH, Köln (DE)

(72) Inventors: Hermann HERTER, Leverkusen (DE); Jaspreet Singh MAROK, Köln (DE); Andrej CEH, Köln (DE)

(73) Assignee: BAYER BUSINESS SERVICES GMBH, Köln (DE)

(21) Appl. No.: 16/320,886

(22) PCT Filed: Jul. 21, 2017
(86) PCT No.: PCT/EP2017/068488

§ 371 Date: Jan. 25, 2019

(30) Foreign Application Priority Data
Jul. 26, 2016 (EP) 16181310.0

(51) Int. Cl. G16H 20/10; G06K 19/07; G06K 19/06; H04L 9/06; G06Q 10/08; G06F 16/28

(52) U.S. Cl. CPC G16H 20/10; G06K 19/0723; G06K 19/06037; G06F 16/282; H04L 9/0643; G06Q 10/087; G06K 19/06028

(57) ABSTRACT

The present disclosure concerns the synchronization of hierarchical data between a sender and a recipient using hash codes. Hierarchical hash codes are used for synchronization of IT systems. The hierarchy of forwarded objects is condensed in the hierarchical hash codes which are exchanged between the IT systems. The complete hierarchy of the underlying and associated child objects is condensed in each hash code of a parent object: identifiers of the child objects and the relationships of the child objects below the parent object with one another and with the parent object. This applies to each aggregation level, so that the hash code of the at least one top element represents the complete hierarchy.

### CROSS REFERENCE TO RELATED APPLICATIONS

This application is a national stage application under 35 U.S.C. § 371 of International Application No. PCT/EP2017/068488, filed internationally on Jul. 21, 2017, which claims priority to European Application No. 16181310.00 filed Jul. 26, 2016.

### FIELD

The present disclosure concerns the synchronization of hierarchical data between a sender and a recipient using hash codes into which the hierarchical structure of the data is incorporated (hierarchical hash codes).

### BACKGROUND

The synchronization of hierarchical data between a sender and a recipient plays an important part in many industrial, logistical and commercial processes.

The tracking of commodities, in particular pharmaceutical products, can be cited as an example. Pharmaceutical products are subject to a series of official regulations worldwide. In some countries, this includes a requirement to ensure the traceability of pharmaceutical products (Track & Trace). For this purpose, individual pharmaceutical packages are provided with a unique identifier (e.g. a serial number) (serialization) so that they can be uniquely identified at a later time.

This identifier is normally an (alpha)numeric code which is usually applied in machine-readable form, for example as a barcode or 2D matrix code, to the pharmaceutical package, or is stored in an RFID transponder which is connected to the pharmaceutical package.

US 2006/0174129A1 discloses a method for authenticating items. Each individual item carries a unique identifier which is stored in an RFID transponder which is connected to the item. A sender generates hash codes from the individual identifiers. The hash codes are forwarded from the sender to a recipient together with or separately from the items. The recipient in turn generates hash codes from the identifiers and can verify the authenticity of the forwarded items by comparing the self-generated hash codes with the hash codes forwarded by the sender. However, the method is restricted to the mapping of parent-child relationships only, so that all parent hash codes must be verified for the full synchronization of a hierarchy.

Pharmaceutical products are not normally sold in the form of individual packages, but rather a plurality of individual packages are combined into larger units (bundles). A plurality of bundles can be combined in a box and a plurality of boxes can be stacked on a pallet. The individual packages in turn similarly carry a unique identifier.

On an abstract level, a hierarchy of objects with a plurality of aggregation levels is present here. FIG. 1 illustrates this by way of example on the basis of a package hierarchy.

The smallest packaging units are referred to as “child objects” or “children”; they are present at the bottom (zero-th) aggregation level (Level 0). They themselves have no child objects.

Objects at the next-highest packaging level at which a plurality of “children” are combined are referred to as “parent objects” or “parents” (Level 1). A plurality of “parents” can in turn again be combined into one unit. These “parents” are in turn “children” of the next-highest unit. Objects at the top aggregation level are referred to here as top element(s), having only “children”, but no parent.

The process of combining a plurality of units into one “higher” unit is also referred to as aggregation. The object in which a plurality of objects are combined is also referred to as an aggregate. The relationship between the objects at different aggregation levels is also referred to as a hierarchy.

Due to the legal obligation (differing according to the country) of the manufacturer of a commodity and/or of the distribution partners involved to be able to trace the handling of the serialized objects (e.g. production, shipping, etc.), the serialization and aggregation data are stored and processed in the corresponding IT systems of the companies involved.

Pharmaceutical products normally pass through a chain of sales partners before they finally reach the patient. A prerequisite of an efficient logistical handling of the products is that the aggregation information is forwarded (electronically) from the preceding distribution partner to each of the distribution partners involved in the chain. Without this information, the current distribution partner would be compelled to re-identify the content of the respective aggregation stage before onward shipment and to do this would have to unpack it down to the bottom packaging level.

In order to guarantee the correctness of the serialization data and aggregation data along the distribution chain, synchronization between the IT systems involved both inside a company and between companies is required. The synchronization should comprise the individual object, including its validity and the associated parent-child relationship.

US 2011/0264629A1 discloses a method for synchronizing datasets between a sender and a plurality of recipients. In this method, the datasets are subdivided into different levels and hash codes are generated for the elements at the individual levels. The hash codes at the individual levels are successively compared during the synchronization of the datasets. However, no information relating to the underlying levels is incorporated into the generation of a hash codes for a higher level.

### SUMMARY

To generalize, the current conventional method for synchronization can be described as follows:
1) The sender (e.g. of a commodity) forwards the aggregation data to the recipient (e.g. of the commodity) at an agreed time. For this purpose, 1 to n aggregation messages are sent.
2) The recipient processes the forwarded messages and carries out a quantity-based comparison of the delivery document, the physical commodity and the number of objects in its IT system.
3) If the recipient transfers the commodities to a next distribution partner, the recipient forwards the corresponding aggregation data of the transferred commodity units to the sender. It must forward the complete hierarchy.

The quantity-based verification specified under point 2) provides o clues whatsoever regarding the correctness of the aggregation; i.e., the parent-child relationships are completely ignored.

In contrast to the highly simplified example in FIG. 1, real hierarchies comprise hundreds or thousands of objects. The data quantities that must be exchanged between the IT systems in order to keep the IT systems synchronized are correspondingly large.

Due to the data quantities, the data are not necessarily exchanged between the distribution partners in a single large message, but in many smaller, self-contained messages. These messages must currently be processed individually.

Along with the high susceptibility to error due to delayed and lost messages, this means a substantial requirement for computer and memory capacities, and also heavy network traffic (LAN and WAN).

A missing or inconsistent message means a discrepancy between the actually packed objects and their digital mapping in the IT systems. In the worst case, this discrepancy is propagated by the entire distribution chain until the first real verification of the commodities and, along with increased expenditure (returns, correction of official documents, etc.) can also have a negative impact on a company's reputation.

There is therefore a need to simplify the synchronization of hierarchical data between a sender and a recipient, not only within a company (serialization data and aggregation data are normally generated on the company's packaging devices and are transmitted to one or more Track&Trace departments/IT systems within the company), but also between companies (as in the case of the delivery of commodities as described above).

This object is achieved by the subject-matter of independent claims 1 and 11. Some embodiments are set out in the dependent claims and in the present description.

A first subject-matter of the present disclosure is therefore a method comprising the following steps:
provision of objects which are present on at least two aggregation levels: a bottom aggregation level, a top aggregation level and optionally further aggregation levels between the bottom and the top aggregation level,
   wherein each object has a unique identifier,
   wherein objects which are combined into one object at a higher aggregation level are referred to as child objects,
   wherein objects with which child objects at a lower aggregation level are associated are referred to as parent objects,
forwarding of the objects from a sender to a recipient,
generation of hash codes by the sender and by the recipient for each parent object on the basis of the associated child objects,
   wherein a hash code is generated on the basis of the unique identifiers of the parent object, and
   a.) on the basis of the unique identifiers of the associated child objects if the child objects are present at the bottom aggregation level,
   b.) on the basis of the hash codes generated for the child objects if the child objects are present at an aggregation level above the bottom aggregation level,
comparison of the hash codes of objects at a higher aggregation level, such as the top aggregation level, generated by the sender and recipient.

The present disclosure includes the use of hierarchical hash codes for the synchronization of IT systems. The hierarchy of forwarded objects is condensed in the hierarchical hash codes which are exchanged between the IT systems. The complete hierarchy of the underlying and associated child objects is condensed in each hash code of a parent object: identifiers of the child objects and the relationships of the child objects below the parent object with one another and with the parent object. This applies to each aggregation level, so that the hash code of the at least one top element represents the complete hierarchy.

The present disclosure thus offers the facility to check the completeness and correctness of the forwarding of objects and their aggregation relationships quickly and efficiently on the basis of hash codes. In comparison with the methods described in the prior art, it suffices in this case to compare the hash value of the top element in order to check for completeness and correctness.

### BRIEF DESCRIPTION OF THE DRAWINGS

In the drawings:

FIG. 1 shows an example of a hierarchy with different packaging levels.

FIG. 2 shows an example of a generalized hierarchy of objects with three aggregation levels.

FIG. 3 shows an example of the generation of hash codes using a method according to one embodiment.

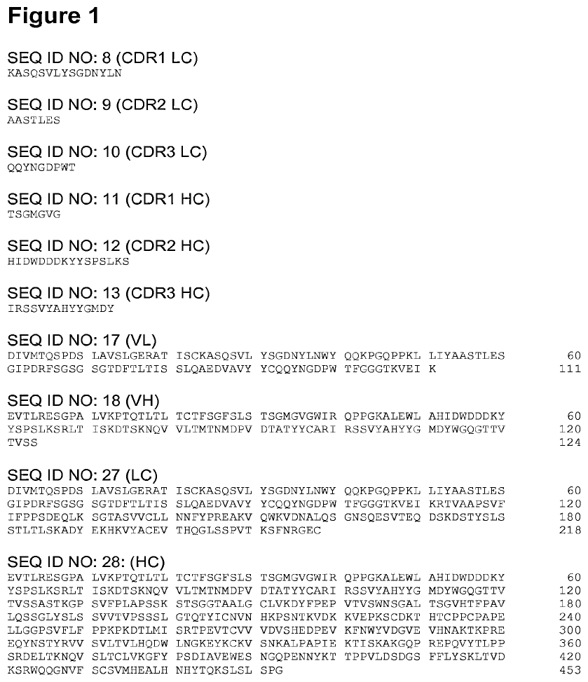

### DETAILED DESCRIPTION

A hash code is the result of a mathematical function, referred to as the hash function. Hash functions are sufficiently known, for example, from cryptography.

A hash function is a mapping which maps an input quantity onto a target quantity (the hash code). The term hash algorithm is also used, especially in information technology, since hash functions are often specified in the form of an algorithm instead of a mathematical function. The input quantity is normally substantially greater than the target quantity, so that the data quantity is reduced when a hash code is generated. The hash code generation process is non-reversible, i.e. the incorporated data can no longer be reproduced from the hash code. A modification of the data being incorporated results in a modification of the resulting hash code.

Examples of hash functions (hash algorithms) can be found, for example, in standard works on information technology, such as “Algorithmen and Datenstrukturen” [“Algorithms and Data Structures”] by Thomas Ottmann and Peter Widmayer, Spektrum-Verlag, 5^{th} Edition 2012, ISBN-13: 9783827428035. Corresponding functions can also be found in various commercially available standard solutions, such as, for example, in SAP products the function block CALCULATE_HASH_FOR_RAW which supports two algorithms SHA1 and MD5, or the class CL_ABAP_MESSAGE_DIGEST which additionally supports the SHA256 algorithm.

In the method according to the present disclosure, a recipient receives a plurality of objects from a sender. Two cases can be distinguished:

1) The objects occur on at least two aggregation levels. This means that there is at least one parent object to which one or more child objects are allocated.

2) No parent-child relationships exist between the objects, but rather all objects are child objects only. In such a case, a logical top element can be introduced as a parent in order to create a logical hierarchy. This case 2) can thus be transferred into the above case 1). If no aggregation relationship between the objects therefore exists, the method can still be applied to a logical top element, for example the product batch.

If objects are therefore described below as being present on “at least two aggregation levels”, this includes the case where one level is present with objects which are combined at a higher level into one logical object.

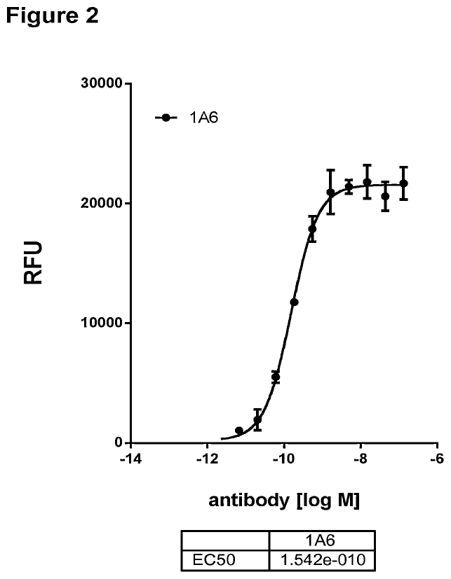

FIG. 2 illustrates the relationships between the objects in a simple hierarchy with three aggregation levels. A plurality of objects (3, 4, 5 and 6) are present at the bottom aggregation level.

The bottom aggregation level will also be referred to below as the zero-th level. The term “zero-th aggregation level” has been chosen because no aggregation of objects takes place at this level, but only at the next-highest, first aggregation level (and then on the basis of the objects at the zero-th aggregation level).

Each object at each aggregation level carries a unique identifier.

A unique identifier is generally understood to mean information which is linked to the object and by means of which an object can be uniquely identified. Even if two objects are regarded by a user as “identical”, i.e. exchangeable, they can be distinguished from one another on the basis of their different identifiers. The identifier is normally an (alpha)numeric code and/or a machine-readable representation of an (alpha)numeric code.

In the example shown in FIG. 2, the objects carry the unique identification numbers 1, 2, 3, 4, 5 and 6 as unique identifiers. Further examples of identifiers are given by way of example in the text below.

In FIG. 2, the child objects 3, 4 and 5 are combined into one parent object 2. The objects 2 and 6 are furthermore combined into one parent object 1. The objects at the bottom (zero-th) aggregation level are thus child objects only, while the objects at the top aggregation level (here the top element 1) are parent objects only.

In the case of a number n of aggregation levels, there are therefore a bottom level and (n-1) higher levels. Of the (n-1) higher aggregation levels, the highest aggregation level is the top aggregation level. The objects at the top aggregation level are also referred to here as top elements. There are (n-2) further aggregation levels between the bottom and the top aggregation level.

The objects present at the bottom aggregation level are child objects only. The objects at the top aggregation level are parent objects only. It is conceivable for only one parent object to be present at the top aggregation level (one top element). However, it is also conceivable for a plurality of parent objects to be present at the top aggregation level (a plurality of top elements). All objects at aggregation levels between the bottom and the top aggregation level are both child and parent objects.

As already described, the at least one top element may also be a logical element, i.e. a unique identifier only. Although the objects are normally physical objects, it is therefore conceivable for a plurality of (physical) objects not to be combined into one higher physical unit at the top aggregation level, but only to be logically connected/linked to one another by a common unique identifier.

One or more hash codes in which the complete hierarchy of the forwarded objects is condensed are generated on both the sender side and the recipient side.

The hash code is generated on the basis of the unique identifiers of the forwarded objects and on the basis of the existing hierarchy. The information indicating the objects which have been forwarded (unique identifiers) and also the hierarchical relationships which exist between the objects (hierarchy) is incorporated into the hash code.

If, for example, an incorrect object is forwarded during the forwarding of the objects from the sender to the recipient, different identifiers for the respective object will be present with the sender and with the recipient and the hash code generated by the sender will not match the hash code generated by the recipient.

If, for example, an object is forgotten during the forwarding of the objects from the sender to the recipient, different hierarchies will be present with the sender and with the recipient and the hash code generated by the sender will not match the hash code generated by the recipient.

The hash code for the forwarded objects thus represents the entire information relating to the forwarded objects and their relationships with one another. If the recipient knows all of the objects involved (for example because they were originally dispatched by it), it suffices to transmit only the hash code of the at least one top element rather than all identifiers of the forwarded objects between the sender and recipient in order to be able to decide, by means of a comparison with the self-generated hash code, whether the forwarding was complete and correct.

As a result, fewer data need to be transferred between the sender and recipient and fewer comparisons need to be performed.

The method according to the present disclosure can be used to synchronize the IT systems of a sender and a recipient in respect of data relating to a hierarchy of objects which have been forwarded from the sender to the recipient. In some embodiments, the method according to the present disclosure comprises the following steps:
forwarding of the objects by the sender to the recipient,
forwarding of information relating to the forwarded objects from the sender to the recipient, wherein the information comprises the unique identifiers of the forwarded objects and the hierarchical relationships between the forwarded objects,
generation of a hash code for each object at the top aggregation level by the sender and by the recipient, wherein the hash code is generated on the basis of the identifiers of the forwarded objects and on the basis of the hierarchical relationships between the forwarded objects,
comparison of the hash code(s) generated by the sender with the hash code(s) generated by the recipient.

The comparison of the hash codes serves to synchronize the IT systems of the sender and recipient. A comparison of this type is performed for different purposes, some of which are detailed below.

One possible purpose is the verification of forwarded aggregation data.

For this purpose, a sender forwards objects which are present on at least two aggregation levels to a recipient in a first step. The sender furthermore forwards the information relating to the forwarded objects, including the existing hierarchy, to the recipient in one or in more, for example electronic, messages. This information is also referred to here as aggregation data.

The sender generates a hash code for each parent object in which the aggregation data are condensed.

The recipient receives the (for example, electronic) messages from the sender and stores them in its IT system. It also receives the objects forwarded by the sender.

In order to then check whether it has correctly received all messages, it similarly generates a hash code for each parent object in which the aggregation data are condensed.

By comparing the hash codes generated by the sender for the top elements with the hash codes generated by the recipient for the top elements, it is possible to recognize whether all messages from the sender have been correctly received by the recipient.

The sender can forward the hash codes generated by it for the top elements to the recipient and the recipient can perform the comparison, or the recipient can forward the hash codes generated by it for the top elements to the sender and the sender can perform the comparison, or the sender and the recipient make the generated hash codes for the top elements mutually available to one another so that both can perform the comparison.

The aforementioned method can be summarized as follows:

verification of forwarded aggregation data, comprising the following steps:
forwarding of objects by a sender to a recipient, wherein the objects are present on at least two aggregation levels: a bottom aggregation level, a top aggregation level and optionally further aggregation levels between the bottom and the top aggregation level, and wherein each object has a unique identifier,
forwarding of information relating to the forwarded objects from the sender to the recipient, wherein the information comprises the unique identifiers of the forwarded objects and the hierarchical relationships between the forwarded objects,
generation of a hash code for each object at the top aggregation level by the sender and by the recipient, wherein the hash code is generated on the basis of the identifiers of the forwarded objects and on the basis of the hierarchical relationships between the forwarded objects,
comparison of the hash code generated by the sender with the hash code generated by the recipient.

A further possible purpose is the acknowledgement of receipt of objects.

For this purpose, a sender forwards objects which are present on at least two aggregation levels to a recipient in a first step. All objects carry a unique identifier.

The sender generates one or more hash codes for the forwarded objects, wherein the unique identifiers of the objects and their hierarchical relationships with one another are incorporated into the generation of the hash codes (or the sender generates the corresponding hash codes before forwarding the objects).

The recipient receives the objects and similarly generates one or more hash codes on the basis of the unique identifiers and the hierarchical relationships between the objects.

The recipient acknowledges receipt of the objects by forwarding the generated hash code(s) to the sender.

By comparing the hash codes generated by the sender and the recipient, the sender can recognize whether all objects have been received by the recipient.

However, it is also conceivable for the sender to forward the hash code(s) generated by it to the recipient so that the recipient can recognize whether all objects which the sender has forwarded have also been correctly received by the recipient.

It is furthermore conceivable for the sender and recipient to make the generated hash codes mutually available to one another.

The aforementioned method can be summarized as follows:

verification of the correct forwarding of objects from a sender to a recipient, comprising the following steps:
provision of objects, wherein each object carries a unique identifier and a plurality of objects (child objects) can be combined on at least one aggregation level into at least one higher-level top element,
forwarding of the objects from the sender to the recipient,
generation by the sender and by the recipient of a hash code for the at least one top element on the basis of the identifiers of the objects and on the basis of the hierarchy of the objects at the aggregation levels,
comparison of the hash code generated by the sender with the hash code generated by the recipient.

A further possible purpose is the forwarding of a modified object inventory.

In a first step, a sender forwards objects which are present on at least two aggregation levels to a first recipient. All objects carry a unique identifier.

Both the sender and the first recipient map in their respective IT system that the objects are now with the recipient.

The first recipient forwards a portion of the objects (wherein “a portion of the objects” can also mean “all objects”) to a second recipient.

The first recipient and/or the second recipient generate one or more hash codes for the forwarded objects, wherein the hash codes are generated on the basis of the unique identifiers of the forwarded objects and on the basis of the hierarchical relationships between the objects.

The first recipient and/or the second recipient forward(s) the generated hash codes for the top elements together with the identifiers of the top elements to the sender. The sender similarly generates the corresponding hash codes for the top elements. If the hash codes match one another, it is ensured that the previous aggregation of the sender is currently still valid and the sender can determine, via its own data inventory, all objects involved which have been forwarded to the second recipient, and it can update its IT system accordingly, since a portion of the object inventory is now no longer stored with the first recipient but with the second recipient. If the hash codes differ from one another, the sender knows that the information is no longer synchronous between it and the first recipient and can initiate corresponding steps to restore the synchronism.

The aforementioned method can be summarized as follows:

forwarding of a modified object inventory in which a first receiver forwards a portion of objects which the first receiver has received from a sender to a second recipient,
wherein each forwarded object carries a unique identifier,
wherein the forwarded objects are present on at least two aggregation levels: a bottom aggregation level, a top aggregation level and optionally further aggregation levels between the bottom and the top aggregation level,

characterized in that the first recipient generates a hash code for each forwarded object at the top aggregation level,
wherein the hash code is generated on the basis of the identifiers of the forwarded objects and on the basis of the hierarchical relationships between the forwarded objects, and

the first recipient or the second recipient forwards the generated hash code to the sender.

A further subject-matter of the present disclosure is a computer program product which can be loaded into the main memory of a computer and which comprises computer program segments which prompt the computer to calculate a hash code for a hierarchy of unique identifiers of objects on the basis of the unique identifiers of the objects and on the basis of the hierarchical relationships between the objects.

The teachings of the present disclosure are explained in detail below without differentiating between the subject-matter (method, computer program product). On the contrary, the explanations below are intended, in some embodiments, to apply accordingly to all subject-matter, regardless of the context (method, computer program product) in which they are given.

The present disclosure focuses on the generation of one or more hierarchical hash codes. The identifiers of the forwarded objects and also the hierarchical relationships between them are incorporated into the generation of a hierarchical hash code, as described below.

A hierarchy of objects, i.e. a plurality of objects, which occur on at least two aggregation levels is transferred.

There is at least one top element (the at least one top element) at the top aggregation level.

Only child objects with their unique identifiers are present at the zero-th aggregation level.

At least one child object at the zero-th aggregation level is allocated to a parent object at the first aggregation level.

A hash code is generated for each parent object in the hierarchy.

The process of generating the hash codes, in some embodiments, starts for a parent object at the first aggregation level. The child objects which are assigned to this parent object are first determined (the process can obviously also be performed in principle from the perspective of the child objects at the zero-th aggregation level: the child objects which are combined at a higher aggregation level into one parent object are determined; however, the process is described below from the perspective of the parent objects for reasons of clarity).

The identifiers of the objects are incorporated into the hash code. All identifiers of those child objects at the zero-th aggregation level which belong to a parent object at the first aggregation level are therefore determined and listed. In addition, the identifier of the parent object is also determined. A first hash code is then generated into which the identifiers of the child objects and the identifier of the parent object are incorporated.

As is generally known, the hash code generation is input-sensitive and is therefore dependent on the sequence of the identifiers which are incorporated into the generation of the hash code. In order to ensure comparability, a unique sequence is first established for the unique identifiers of the associated children (such as by means of a sorting function). The parent identifier is, in some embodiments, not taken into account in the sorting, but may be transferred first. However, it is also conceivable to place the parent identifier at the end of the sequence or to nest the identifiers of the objects involved within one another. It is important that the sequence is defined and that the sender and recipient have agreed in advance on the manner in which the hash code is generated.

In some embodiments, sorting functions are used in order to generate a defined sequence of the identifiers of the objects involved. Sorting functions or sorting methods are algorithms which serve to sort a tuple. If the data to be sorted are words, they can be sorted alphabetically, as in the case of a dictionary, for example. If the data to be sorted are numbers, they can be sorted according to their numeric value (from the lowest number up to the highest number, or vice versa). Alphanumeric character strings can be sorted, for example, on the basis of the ASCII or ANSI code.

If the identifiers of the associated children are arranged in a unique sequence and if the identifier of the corresponding parent object is placed, for example, before or after this sequence, the data string generated in this way is transferred to the hash algorithm.

The result is a first hash code for a first parent object at the first aggregation level into which the identifier of the parent object, the identifiers of the associated child objects and the hierarchical relationship between the objects are incorporated.

If there are further parent objects at the first aggregation level, the corresponding hash codes are formed for all further parents at the first aggregation level on the basis of their own identifiers and the unique identifiers of the associated child objects at the zero-th aggregation level.

A (unique) hash code is thus obtained for each parent object at the first aggregation level.

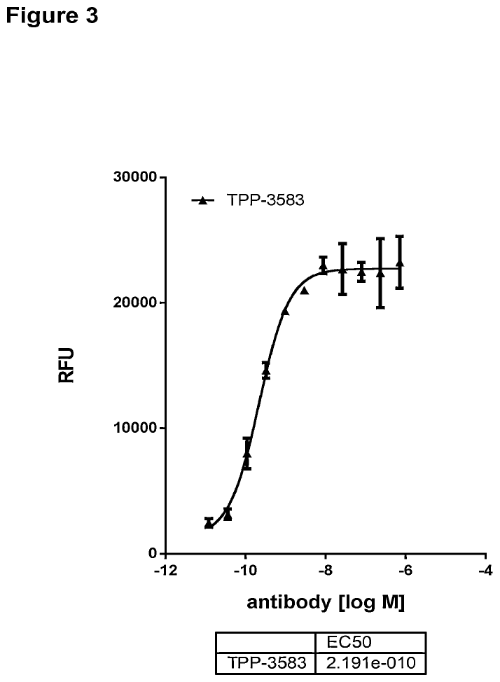

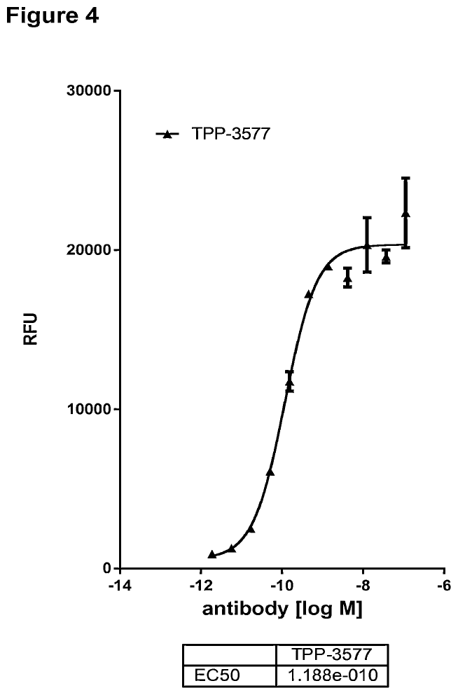

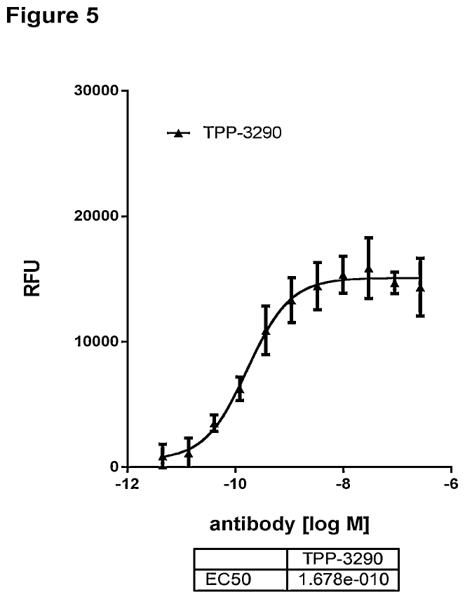

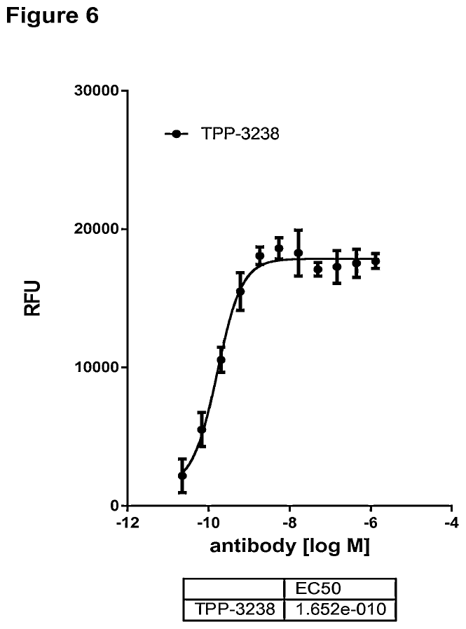

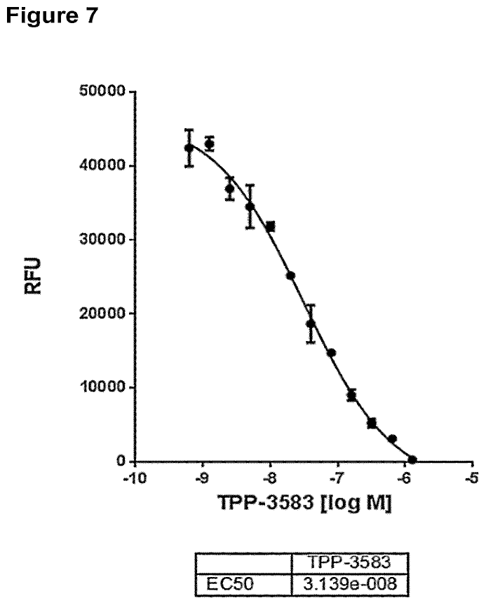

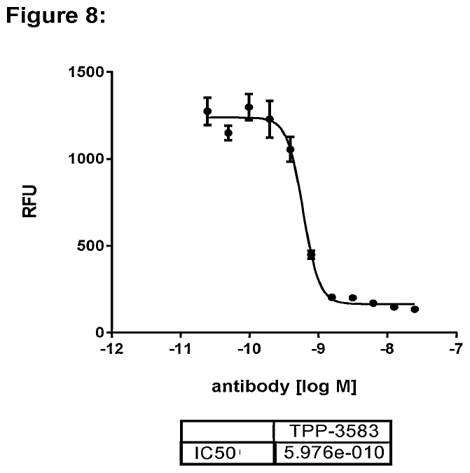

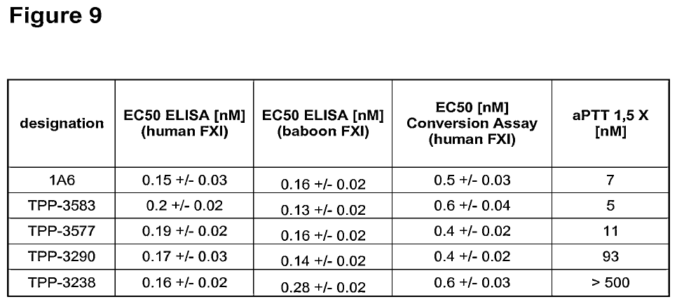

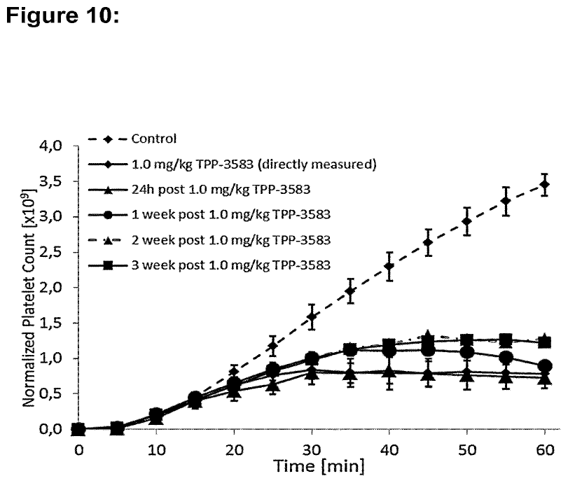

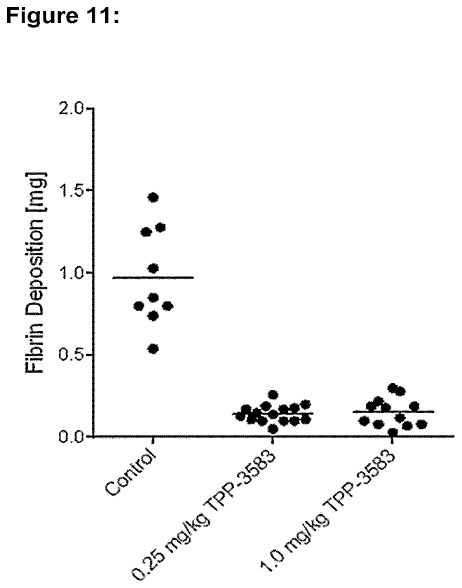

The process is continued if there are one or more further aggregation levels above the first aggregation level. Any parent object at the second aggregation level is picked out and the hash code for this parent object is generated on the basis of its own identifier and on the basis of information relating to the associated child objects at one or more lower aggregation levels. However, unlike the parent objects at the first aggregation level, the hash code for the parent objects at the second (and each further) aggregation level is generated on the basis of the (unique) hash codes generated for the associated child objects (which in turn are after all parent objects also), rather than on the basis of the identifiers of the child objects. This is illustrated, for example, in FIG. 3 (in this respect, see also the description of FIG. 3 further below).

Before the hash function is applied, a unique sequencing of the hash codes of the child objects is also carried out (e.g. by means of sorting) in the case of the generation of a hash code for a parent object on the basis of its identifier and the hash codes of the associated child objects.

The process continues within the existing hierarchy from bottom to top until a corresponding hash code has been generated for each parent object. Finally, the hash code is generated for the at least one top element (if a plurality of top elements are present, a hash code is generated for each top element).

To generalize, this produces the following traversing through the aggregation tree: If C are the children of a parent P, this results in a post-order traversing of the form C-P, starting with the top elements. The recursive pass ensures that all hash values at the underlying aggregation levels are first determined.

The particular feature of the hash code of a parent object is that it reflects the entire hierarchy below the parent object and the allocation to the parent object itself.

A change in the hierarchy, for example the removal of a child object, results in a change in the hash code H₁ of the parent object into which the identifier of the child object (or the hash code of the child object if the child object in turn is a parent object) is incorporated. However, the hash code H₂ of the parent object at the next-highest aggregation level into which the hash code H₁ is incorporated therefore also changes, and so on.

The hash code of a parent object thus represents the entire underlying hierarchy. In order to check whether objects which have been forwarded from a sender to a recipient have also been forwarded correctly, it is not necessary according to the present disclosure for each individual object to send back an identifier for the check. It suffices to compare the hash codes at the top aggregation level (the hash codes of the top elements). If they match one another, all objects which have been dispatched by the sender have also been received by the recipient or all changes in the hierarchy have been verified. If they do not match one another, the shipment is incorrect and the error search can begin. The hash codes at the next-lowest aggregation level can be compared during this error search. The aggregates have been forwarded correctly in the case of all hash codes which match one another. In the case of all hash codes which do not match one another, one or more objects have not been forwarded or have been forwarded with a different identifier. It is also conceivable that objects have been forwarded which the sender has not recorded. The comparison of the hash codes is continued “downward” (from the top in the direction of the bottom aggregation level) until all correctly shipped aggregates and objects and all errors have been determined.

In some embodiments, the described method according to the present disclosure for generating the hierarchical hash codes can be summarized as follows:
provision of a hierarchy of objects on at least two aggregation levels: a bottom aggregation level and a top aggregation level, and optionally further aggregation levels between the bottom and the top aggregation level,
   wherein each object carries a unique identifier,
   wherein objects which are combined into one object at a higher aggregation level are referred to as child objects,
   wherein objects with which child objects at a lower aggregation level are associated are referred to as parent objects,
forwarding of the objects from a sender to a recipient,
generation of hash codes by the sender and by the recipient for each parent object on the basis of the associated child objects,
   wherein a hash code is generated on the basis of the unique identifiers of the parent object, and
   a.) on the basis of the unique identifiers of the associated child objects if the child objects are present at the bottom aggregation level,
   b.) on the basis of the hash codes generated for the child objects if the child objects are present at an aggregation level above the bottom aggregation level,
   wherein, before a hash code is generated, the identifiers incorporated into the generation of the hash code and incorporated hash codes are arranged in a defined sequence,
comparison of the hash codes of the objects at a higher aggregation level, such as the top aggregation level, generated by the sender and recipient.

FIG. 3 shows by way of example the generation of the hash codes for all objects in a hierarchy. MLHC stands for “Multi Level Hierarchy Code” and designates the generated hierarchical hash code.

Six individual objects are located at the lowest (zero-th) aggregation level. They carry only one digit as a unique identifier in order to simplify the illustration. The first object seen from left to right at the bottom level carries the unique identifier 1. The next object seen from left to right carries the unique identifier 2. The objects with the unique identifiers 4, 3, 8 and 9 then follow.

The objects with the identifiers 1 and 2 are combined into one aggregate at the first aggregation level. This aggregate similarly carries a unique identifier, the digit 5. The objects with the identifiers 3 and 4 are similarly combined into one aggregate with the digit 6.

The objects with the identifiers 8 and 9 are combined into one aggregate 10 at level 2.

Aggregates 5 and 6 are combined into one aggregate 7 at the next-highest aggregation level. Aggregates 7 and 10 are combined into one aggregate 11 at the top aggregation level. Object 11 is thus the top element in the hierarchy.

If the hierarchy 11->7->(5, 6)->(1, 2) is compared with the hierarchy 11->10->(8, 9), it is evident that the hierarchy 11->7->(5, 6)->(1, 2) has one aggregation level more than the hierarchy 11->10->(8, 9). This, however, is irrelevant. In some embodiments, the method according to the disclosure also encompasses mixed hierarchies of this type.

The hash code generation begins with an aggregate at the first aggregation level. In the case of the hierarchy 11->7->(5, 6)->(1, 2), this is aggregate 5 or aggregate 6. The hash code MLHC(5) of aggregate 5 is generated on the basis of its own identifier (5) and the identifiers 1 and 2. The hash code MLHC(6) of aggregate 6 is generated on the basis of its own identifier (6) and the identifiers 3 and 4. The identification numbers (identifiers) are arranged in ascending order before the hash codes are generated. To illustrate the sorting sequence, object 4 is located to the left of object 3; whereas, in the sorted sequence, the identification number 3 is located to the left of the identification number 4: MLHC(6)=hash(6, 3, 4).

The sorted identification numbers of the children are prefixed in each case by the identifier of the parent.

The hash code MLHC(7) of aggregate 7 is generated on the basis of its own identifier (7) and the hash codes MLHC(5) and MLHC(6).

The hash code MLHC(10) of aggregate 10 is generated on the basis of its own identifier and the identifiers 8 and 9.

The hash code MLHC(11) of aggregate 11 is generated on the basis of its own identifier and the hash codes MLHC(7) and MLHC(10).

Who performs the comparison of the hash codes is irrelevant to the present disclosure.

In one embodiment, the recipient acknowledges receipt of the objects from the sender by forwarding the generated hash code(s) at the top aggregation level to the sender. The sender compares the hash code(s) generated by the recipient with the corresponding hash codes which the sender itself has generated.

In a different embodiment, the sender forwards the objects and the corresponding generated hash codes. The recipient in turn generates the hash codes and, by comparing the self-generated hash codes with the forwarded hash codes, checks whether all objects have been forwarded (completeness) and whether the correct objects have been forwarded (correctness).

In a different embodiment, the sender and recipient forward the generated hash codes reciprocally to one another. Both are capable of performing a comparison of the hash codes and in this way synchronize their IT systems.

The present disclosure can be used in principle for any objects. The objects may be physical objects or digital objects.

Examples of digital objects are text files, image files, music files, video files and the like, the hierarchy of which is defined by the directory structure or other logical top elements. Examples of physical objects are commodities. Living beings such as plants, animals and people can also be objects within the meaning of the present disclosure.

In one embodiment, the objects are physical (i.e. physical or tangible) objects. Some physical objects are commodities.

The term commodity is understood to mean movable items which are or may be the subject of commercial traffic. Some commodities within the meaning of the present disclosure are those for which traceability is required by national authorities and/or by law.

Some commodities are pharmaceutical products (drugs, medicines, diagnostic products and others).

Commodities are normally present at different aggregation levels in commercial traffic. This can be illustrated by the example of a medicine. The smallest unit in which medicines are normally forwarded in commercial traffic is a medicine package in which the medicine (e.g. a blister pack containing tablets) is stored together with a package insert. The medicine packet is normally a folding cardboard package. However, no individual medicine packages are transported in commercial traffic from the manufacturer to a distribution station or from one distribution station to the next distribution station. Instead, a plurality of medicine packages are combined, for example, into a shipping box.

Individual medicine packages can be regarded as the zero-th aggregation level, and a bundle of medicine packages can be regarded as the first aggregation level. In principle, the individual tablets which are combined in a blister pack could already be understood as the first aggregation level. However, since medicines are not normally sold in commercial traffic in the form of individual tablets, it is more appropriate, for example, to define the shipping box as the first aggregation level. The shipping box is regarded below as the first aggregation level by way of example, without limiting the method.

The shipping boxes consisting of combined medicine packages can in turn be combined into one higher aggregation unit. A plurality of shipping boxes can be stored, for example, on a pallet. The pallet thus represents a further aggregation unit at a further aggregation level.

According to the disclosure, the individual commodities are provided with a unique identifier at all aggregation levels. This means that even the smallest commodity occurring as a unit in commercial traffic carries an identifier which distinguishes it from other units. When applied to the example of medicine packages, this means that one medicine package carries a unique identifier which distinguishes it from a second medicine package.

An identifier is, in some embodiments, understood to mean a string of characters and/or digits which can, in principle, be made accessible to machine-based data processing. The identifier may be a machine-readable representation of a string of characters and/or digits.

An identifier could accordingly be a string of digits. The digit string of a first commodity differs from the digit string of a second commodity, whereas the first and second commodity are regarded by a consumer or user as interchangeable.

The identifier, in some embodiments, occurs in a form in which it is accessible to a machine-based recording. One example is an optically readable code. Widespread forms of optical codes are barcodes or 2D codes. Examples of barcodes are “Codabar” and “Code 128”; examples of 2D codes are “Aztec code”, “Matrix code” and “QR code”.

An optically readable code is normally applied to the packaging of the commodity.

However, the identifier can also be stored in the memory of an RFID transponder which is connected to the commodity. Further identifiers are conceivable.

In some embodiments, an optically readable code is used an identifier for the unique marking (e.g. serialization) of the individual commodities. A 2D code may be used, such as a 2D Matrix code.

Along with the individual commodities, the aggregation units at the higher aggregation levels are also provided with unique identifiers (e.g. SSCC=Serial Shipping Container Code).

Commodities normally pass through a plurality of distribution stations before they reach their final destination. In the case of the medicine packages, they are distributed by the manufacturer via wholesalers and intermediaries to doctors, hospitals and pharmacies before they finally reach the patient.

In a distribution chain of this type, there is therefore a first distribution station (sender) from which the commodities are transported to a second distribution station (recipient). The first distribution station may be a manufacturer of the commodities. It may, however, also be a company which packages the commodities. A wholesaler may also be the first distribution station within the meaning of this disclosure.

The transportation of commodities within a company can also be verified by means of some methods according to the disclosure. In such a case, the first distribution station (sender) is one department within a company by which commodities are forwarded to another department (recipient) of the same company.

The first distribution station is, in some embodiments, the organization which is subject to legal or official regulations governing the traceability of the commodities. The first distribution station is therefore, in some embodiments, the distribution station which may submit proof to an authority or other national organization of the origin and destination of the commodities.

The second distribution station is a distribution station which receives commodities (recipient) and which is located in the logistics supply chain downstream of the first distribution station.

This may be the distribution station which immediately follows the first distribution station in the distribution chain; however, it is also conceivable for one or more further distribution stations to be located between the first and the second distribution station.

The first distribution station therefore transports commodities to the second distribution station, either directly or via one or more further distribution stations.

The second distribution station receives and records the commodities. Recording is understood to mean that all incoming commodities are registered. This is normally done by reading the identifiers of the highest aggregation levels, such as, for example, from the pallets into a computer system.

In the case of optical codes, the individual optical codes on the aggregation units and on the individual commodities are read using an optical scanner. If RFID transponders are used, the identifiers of all RFID transponders are read using a corresponding reading device. The data which are read out are recorded and stored in a computer system.

Both the first and the second distribution station generate the hash codes for the forwarded commodities. To do this, the unique identifiers at the zero-th aggregation level (medicine packages) are determined, in some embodiments sorted and fed to a hash algorithm which generates a unique hash code. If more than two aggregation levels are present, the hash codes of the higher packaging units are generated on the basis of the hash codes of the associated lower packaging units.

In one embodiment of the present disclosure, the second distribution station then forwards a message to the first distribution station in order to acknowledge receipt of the commodities (this acknowledgement may be used, for example, as proof in terms of data integrity). The message comprises only the hash code(s) at the top aggregation level.

On the basis of the message, the first distribution station verifies whether all commodities dispatched to the second distribution station have also actually arrived there. To do this, it compares the hash code(s) forwarded by the second distribution station with the corresponding self-generated hash code(s). If they match one another, all data have been received and processed completely and correctly by the second distribution station. This certainty in connection with the inspection of the integrity of the outer packaging, for example shrink wrapping of the pallet, then indicates that the commodity has also been completely received.

As described above, it is obviously also conceivable for the first distribution station to forward the generated hash code(s) to the second distribution station and for the second distribution station to perform the comparison, or for the first and the second distribution station to make the hash codes mutually available to one another in order to synchronize their IT systems.

It is conceivable that the inventory of objects changes continuously and the respective hash values have to be recalculated constantly so that the reality mapped in the IT system also matches the actual reality. Since the constant recalculation of the hash codes may require resources, in one embodiment the recalculation of the hash codes is not performed immediately after an inventory change, but only later, for example only once an hour or only once a day or only if a specific event occurs, for example an object of the hierarchy is scanned in.

However, so that no incorrect mapping of the reality exists in the IT system in the meantime, all of the hash codes which have to be recalculated because changes have occurred in the hierarchy below these hash codes are provided with a digital notification. This notification indicates to the user of the IT system or to the system itself that the hash code is “outdated”, i.e.

is no longer current and cannot therefore be used for comparisons. The notification is removed following a recalculation of the outdated hash codes.

1: A method comprising:
providing a plurality of objects which are present on at least two aggregation levels: a bottom aggregation level, a top aggregation level and optionally further aggregation levels between the bottom and the top aggregation level,
wherein each object has a unique identifier,
wherein objects which are combined into one object at a higher aggregation level are referred to as child objects,
wherein objects with which child objects at a lower aggregation level are associated are referred to as parent objects;
forwarding the objects from a sender to a recipient;
generating hash codes by the sender and by the recipient for each parent object on the basis of the associated child objects,
wherein a hash code is generated on the basis of the unique identifiers of the parent object, and
a.) on the basis of the unique identifiers of the associated child objects if the child objects are present at the bottom aggregation level, and
b.) on the basis of the hash codes generated for the child objects if the child objects are present at an aggregation level above the bottom aggregation level; and
comparing the hash codes of objects at a higher aggregation level, preferably the top aggregation level, generated by the sender and recipient.

2: The method of claim 1, wherein at least a portion of the objects are physical objects.

3: The method of claim 1, wherein there is at least one object at the top aggregation level which is a unique identifier and does not comprise a physical object.

4: The method of claim 1, wherein before a hash code is generated, the identifiers and the already generated hash codes which are incorporated into the generation are arranged in a defined sequence which has been agreed between the sender and recipient.

5: The method of claim 4, wherein, when the hash codes are generated, the identifiers being incorporated and the hash codes being incorporated are sorted in an order selected from ascending order and descending order, and the identifiers of the objects for which the respective hash code is generated are placed in a position selected from before the sorted sequence and after the sorted sequence.

6: The method of claim 1, wherein the objects are commodities.

7: The method of claim 1, wherein the aggregation levels represent one or more of different packaging stages and bundling of the objects.

8: The method of claim 1, wherein it forms part of a process for tracking commodities from a first distribution station to a second distribution station.

9: The method of claim 1, wherein, if an object changes, the hash codes affected by the change are provided with a digital notification that they need to be updated.

10: The method of claim 9, wherein the hash codes are updated at defined times.

11: A non-transitory computer-readable storage medium for generating and comparing hash codes, the non-transitory computer-readable storage medium storing instructions configured to be executed by one or more processors to cause a system to:
provide a plurality of objects which are present on at least two aggregation levels: a bottom aggregation level, a top aggregation level and optionally further aggregation levels between the bottom and the top aggregation level,
wherein each object has a unique identifier,
wherein objects which are combined into one object at a higher aggregation level are referred to as child objects,
wherein objects with which child objects at a lower aggregation level are associated are referred to as parent objects;
forward the objects from a sender to a recipient;
generate hash codes by the sender and by the recipient for each parent object on the basis of the associated child objects,
wherein a hash code is generated on the basis of the unique identifiers of the parent object, and
a.) on the basis of the unique identifiers of the associated child objects if the child objects are present at the bottom aggregation level, and
b.) on the basis of the hash codes generated for the child objects if the child objects are present at an aggregation level above the bottom aggregation level; and
compare the hash codes of objects at a higher aggregation level, preferably the top aggregation level, generated by the sender and recipient.

12: The method of claim 1, wherein the objects are pharmaceutical products.

13: The method as claimed in of claim 9, wherein the hash codes are updated with the occurrence of an event.
